(19) United States
(12) Patent Application Publication
(10) Pub. No.: US 2026/0224221 A1
(43) Pub. Date: Aug. 6, 2026

(54) SEPTAL DEFECT OCCLUSION DEVICE

(71) Applicant: Edwards Lifesciences Corporation, Irvine, CA (US)

(72) Inventors: John J. Desrosiers, San Clemente, CA (US); Nima V. Nia, Mission Viejo, CA (US); Biswa P. Das, Irvine, CA (US); Sandip Vasant Pawar, Irvine, CA (US); Juan Valle Raleigh, Tustin, CA (US)

(21) Appl. No.: 19/636,660

(22) Filed: Apr. 1, 2026

Related U.S. Application Data
(63) Continuation of application No. PCT/US2024/049541, filed on Oct. 2, 2024.
(60) Provisional application No. 63/542,262, filed on Oct. 3, 2023.

(51) Int. Cl. A61B 17/12; A61B 17/00; A61B 90/00

(52) U.S. Cl. CPC A61B 17/12122; A61B 17/00234; A61B 17/12031; A61B 17/1219; A61B 90/39; A61B 2017/00004; A61B 2017/00243; A61B 2017/00292; A61B 2017/1205; A61B 2090/3966

(57) ABSTRACT

An implantable device can be configured to occlude an opening in a septum of a heart, such as a perimembranous ventricular septal defect. The device can comprise an expandable member expandable from a first state to a second state upon contact with a fluid. The implantable device in the second state can be configured to occlude the opening in the septum, thereby preventing blood from leaking through the opening and across the septum. The implantable device can be substantially or completely devoid of metal, resulting in a reduced likelihood that the implantable device affects the function of heart valves near the opening of the septum or leads to tissue erosion.

### CROSS-REFERENCE TO RELATED APPLICATIONS

This application is a continuation of International Application No. PCT/US2024/049541, filed Oct. 2, 2024, which claims the benefit of U.S. patent application Ser. No. 63/542,262, filed on Oct. 3, 2023, the entire disclosures all of which are incorporated by reference for all purposes.

### FIELD

The present disclosure relates to implantable medical devices and methods for repairing septal defects, and in particular ventricular septal defects.

### BACKGROUND

The human heart can suffer from various defects. These defects can result in significant malfunctioning of the heart and ultimately require correction of the defect. One common type of defect can include a ventricular septal defect (which is also referred to herein as a “VSD” and/or a “defect”). One of the most common types of VSDs is a perimembranous VSD, which is a hole in an upper portion of the septum separating the left and right ventricles of the heart. The perimembranous VSD is typically disposed near or adjacent valves of the human heart (for example the tricuspid valve, aortic valve, and/or pulmonary valve). Blood can flow through the perimembranous VSD from the left ventricle to the right ventricle, which can detrimentally lead to extra blood flowing into the lungs. Perimembranous VSDs that do not close spontaneously may predispose a person to later sequelae such as left heart dilation, arrythmia, aortic insufficiency, pulmonary arterial hypertension, and endocarditis.

Accordingly, a need exists for devices and methods for repairing perimembranous ventricular septal defects.

### SUMMARY

Described herein are implantable medical devices, delivery apparatuses, and methods for implanting medical devices. The disclosed implantable medical devices, delivery apparatuses, and methods can, for example, be configured to occlude a perimembranous VSD disposed near a native valve of the human heart without affecting valve function. As such, the devices and methods disclosed herein can, among other things, overcome one or more of the deficiencies of typical implantable occlusion devices. In some examples, implantable medical devices disclosed herein can comprise an expandable non-metal body configured to occlude a perimembranous VSD.

An implantable device can be configured to occlude an opening in a septum of a heart.

In some examples, the device can comprise an expandable member.

In some examples, the expandable member can be configured to expand from a first state to a second state upon contact with a fluid (for example, blood, saline, and/or contrast solution).

In some examples, the expandable member can be substantially devoid of metal.

In some examples, the expandable member can be completely devoid of metal.

In some examples, the expandable member can comprise a hydrogel.

In some examples, the expandable member can comprise a gelatin sponge.

In some examples, the expandable member can be bioabsorbable.

In some examples, the expandable member can be tissue ingrowth promoting.

In some examples, the expandable member can be radiopaque.

In some examples, the expandable member can have a first cross-sectional area in the first state and have a second cross-sectional area in the second state.

In some examples, the second cross-sectional area of the expandable member can be greater than a planar area of the opening in the septum.

In some examples, the implantable device can comprise an expandable container defining a cavity therein and a plurality of expandable beads disposed within the cavity of the expandable container and configured to be hydrated to an expanded state.

In some examples the expandable container can comprise first and second compartments.

In some examples, the first compartment can be configured to be filled with a first number of the plurality of expandable beads and the second compartment can be configured to be filled with a second, lesser number of the plurality of expandable beads.

In some examples, the plurality of expandable beads can comprise a first set of expandable beads having a first size in the expanded state and a second set of expandable beads having a second size in the expanded state.

In some examples, a method of implanting a medical device in a heart can comprise, using a catheter, positioning the medical device at the ventricular septum and advancing a plurality of expandable beads from the catheter into a cavity of the medical device.

In some examples, the method can further comprise, prior to advancing the plurality of expandable beads, determining a number of the plurality of expandable beads to advance from the catheter.

In some examples, the determination can be based on measurements derived from a fluoroscopic image.

In some examples, an implantable device for occluding an opening in a septum of a heart can comprise an expandable member that is configured to expand from a first state to a second state upon contact with a fluid (for example, blood, saline, and/or contrast solution).

In some examples, an implantable device for occluding an opening in a septum of a heart can comprise a non-metal expandable body expandable between a first state and a second state.

In some examples, an implantable medical device for occluding an opening of a ventricular septal defect of a heart can comprise an expandable container defining a cavity therein and a plurality of expandable beads disposed within the cavity of the expandable container and configured to be hydrated to an expanded state.

In some examples, a method of implanting a medical device in a heart can comprise advancing a catheter coupled to the medical device towards a ventricular septal defect of the heart, using the catheter, positioning the medical device at the ventricular septal defect, and advancing a plurality of expandable beads from the catheter into a cavity of the medical device.

In some examples, an implantable medical device can comprise one or more of the components recited in examples below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit
the scope of the claimed subject matter. The foregoing and other objects, features, and advantages of the disclosure will become more apparent from the following detailed description, claims, and accompanying figures.

The above method(s) can be performed on a living animal (including a human) or on a simulation, such as on a cadaver, cadaver heart, anthropomorphic ghost, simulator (for example, with body parts, heart, tissue, etc. being simulated).

### BRIEF DESCRIPTION OF THE DRAWINGS

FIG. 1A is a perspective view of a cross section of a heart showing an implantable medical device implanted in a ventricular septal defect of the heart, according to one example.

FIG. 1B is a side view of an implantable medical device having a “bell” or “mushroom” shape, according to one example.

FIG. 1C is a side view of an implantable medical device having a cylindrical shape, according to one example.

FIG. 1D is a side view of an implantable medical device having an “hourglass” shape, according to one example.

FIG. 1E is a side view of an implantable medical device having a dumbbell shape, according to one example.

FIG. 2A is a perspective view of a cross section of a heart showing an implantable medical device implanted in a ventricular septal defect of the heart, according to one example.

FIG. 2B is a side view of an implantable medical device, according to one example.

FIG. 3 is a perspective view of a cross section of a heart showing a delivery apparatus and an implantable medical device, according to one example.

FIG. 4 is a perspective view of a fabric that can be used as a covering for an implantable medical device, according to one example.

FIG. 5 is a schematic of a knitting pattern for a fabric that can be used as of a covering for an implantable medical device, according to one example.

FIG. 6 is a schematic of a knitting pattern for a fabric that can be used as a covering for an implantable medical device, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a
device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, the term “axial direction” refers to a direction that is parallel to a central longitudinal axis of an object, such as a delivery apparatus or an implantable medical device.

As used herein, the terms “radial direction” and “lateral direction” refer to a direction that extends radially outward from the central longitudinal axis of the object and is perpendicular to the axial direction.

As used herein, the term “circumferential direction” refers to a direction along a circumference of the object.

As used herein, the term “thickness” refers to a radial thickness of the object between an inner circumferential surface and an outer circumferential surface of the object.

As used herein, “e.g.” means “for example,” and “i.e.” means “that is.”

### Overview of the Disclosed Technology

In some examples, a typical method for repairing a perimembranous ventricular septal defect (“VSD,” which is also referred to herein as a “defect”) can comprise a transcatheter repair procedure in which an implantable medical device is delivered to an implantation site at the VSD through the subject's vasculature and subsequently expanded at the implantation site to cover or occlude the VSD. As opposed to a typical surgical repair procedure, a typical transcatheter repair procedure can be minimally invasive, thereby beneficially resulting in faster patient recovery and fewer potential complications. However, in some examples, the perimembranous VSD may not be a favorable candidate for the transcatheter repair procedure due to the VSD's proximity to an anatomical structure, such as a heart valve.

In some examples, a medical device used in a transcatheter repair procedure can be deployed at the VSD and can be subsequently expanded to cover or occlude the VSD. The medical device typically comprises a metal frame. If the medical device is improperly positioned, the metal frame of the medical device can partially or completely block the heart valve. In some examples, the metal frame can comprise an offset feature (such as an aperture or an opening) oriented closest to the heart valve to mitigate the risk of the metal frame blocking the heart valve. The offset feature can allow blood to flow through the heart valve and/or ensure that the metal frame does not otherwise interact with the heart valve. However, the medical device must be clocked to align the offset feature of the metal frame with the valve.

To address one or more problems of typical implantable medical devices, it would be desirable to develop an implantable medical device capable of occluding a VSD (for example, a perimembranous VSD) without affecting the function of nearby heart valves. Furthermore, it would be desirable to develop an implantable medical device that does not require clocking prior to or during implantation. Finally, it would be desirable to develop an implantable medical device that further reduces the likelihood of septal tissue erosion and heart block.

### Examples of the Disclosed Technology

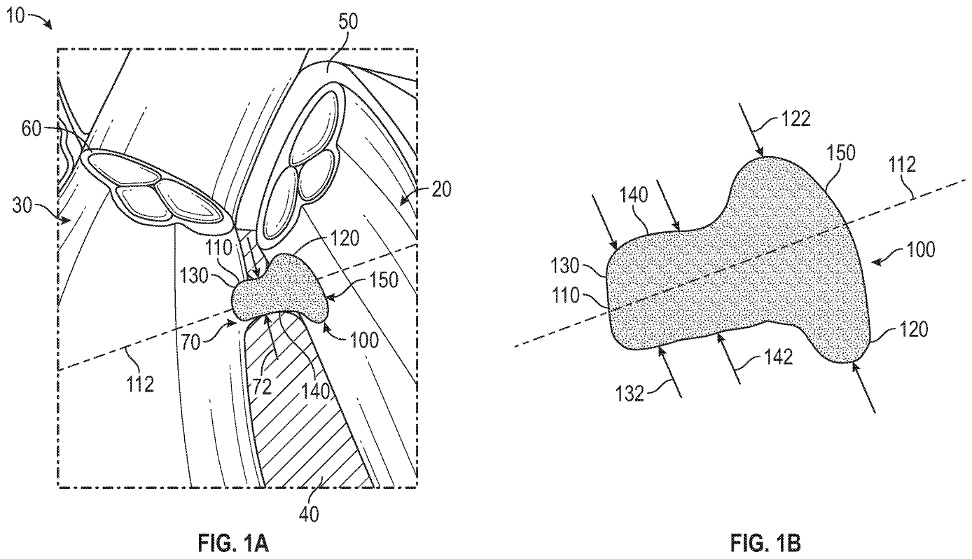

FIG. 1A is a perspective view of a cross section of a heart 10 showing an implantable medical device 100 implanted in a ventricular septal defect 70 of the heart 10, according to one example. The heart 10 comprises a left ventricle 20, a right ventricle 30, a septum 40 separating the left ventricle 20 and the right ventricle 30, an aortic valve 50, and a pulmonary valve 60. The mitral valve, tricuspid valve and the left and right atria of the heart are not shown in FIG. 1A. When the heart 10 contracts during systole, oxygenated blood flows from the left ventricle 20 through the aortic valve 50 to the body and deoxygenated blood flows from the right ventricle 30 through the pulmonary valve 60 to the lungs. During diastole, oxygenated blood from the left atrium flows into the left ventricle 20 and deoxygenated blood from right atrium flows into the right ventricle 30.

The ventricular septal defect 70 (which is also referred to herein as a “VSD” or a “defect”) comprises a hole or an opening in the septum 40 through which blood can flow,

typically from the left ventricle 20 to the right ventricle 30. The hole or opening of the VSD 70 can define a VSD planar area 72 (which is also referred to herein as a “defect cross-sectional area,” “defect diameter,” an “opening planar area,” and/or an “opening cross-sectional area”). As shown, the VSD 70 can be a perimembranous ventricular septal defect adjacent or nearby at least one of the aortic valve 50 and the pulmonary valve 60. In some examples, the VSD 70 can be any one of a muscular ventricular septal defect, an inlet ventricular septal defect, or a conoventricular ventricular septal defect.

The implantable medical device 100 (which is also referred to herein as a “medical device” or an “occlusion device”) can comprise an expandable member 110 (which is also referred to herein as an “expandable body” and/or an “expandable structure”). The expandable member 110 can comprise a structure or body formed from a hydration-expandable material and can be expandable between a first state (which is also referred to herein as a “compressed state,” a “dehydrated state,” and/or a “delivery state”) and a second state (which can also be referred to herein as an “expanded state,” a “hydrated state,” and/or a “deployed state”). The expandable member 110 can be in the first state prior to implantation in the subject's heart 10 and, as shown in the illustrated example, can be in the second state after the implantable medical device 100 has been implanted in the VSD 70.

The medical device 100 can define a central longitudinal axis 112 extending along a length of the medical device 100. The central longitudinal axis 112 can be substantially perpendicular to a plane formed by the septum 40 and/or extend through the VSD 70 from the left ventricle 20 to the right ventricle 30 when the medical device 100 is implanted at the VSD 70. The expandable member 110 can comprise a left ventricular portion 120, a right ventricular portion 130, and an intermediate portion 140 aligned along the central longitudinal axis 112. The left ventricular portion 120 can be configured to be disposed in the left ventricle 20 when the expandable member 110 is in the second state and implanted in the heart 10. The right ventricular portion 130 can be configured to be disposed in the right ventricle 30 when the expandable member 110 is in the second state and implanted in the heart 10. The intermediate portion 140 can be disposed between the left ventricular portion 120 and the right ventricular portion 130 of the expandable member 110.

As shown in FIG. 1B, when the medical device is in the second state, the left ventricular portion 120 can define a left ventricular portion cross-sectional area 122, the right ventricular portion 130 can define a right ventricular portion cross-sectional area 132, and the intermediate portion can define an intermediate portion cross-sectional area 142. Each cross-sectional area 122, 132, and 142 can extend in a radial direction substantially perpendicular to the central longitudinal axis 112 of the medical device 100. The left ventricular portion cross-sectional area 122, the right ventricular portion cross-sectional area 132, and the intermediate portion cross-sectional area 142 can be the largest cross-sectional area of the left ventricular portion 120, the right ventricular portion 130, and the intermediate portion 140, respectively.

As shown in FIG. 1B, in some examples, when the expandable member 110 is in the second state, it can have an asymmetric shape, such as a bell or mushroom shape, wherein the left ventricular portion cross-sectional area 122 can be greater than each of the right ventricular portion cross-sectional area 132 and the intermediate portion cross-sectional area 142. As shown, the right ventricular portion cross-sectional area 132 can be equal to the intermediate portion cross-sectional area 142. However, in some examples, the right ventricular portion cross-sectional area 132 can be less than the intermediate portion cross-sectional area 142. As shown, when the expandable member 110 in the bell or mushroom shape, the intermediate portion 140 can taper from the left ventricular portion 120 to the right ventricular portion 130. In some examples, the intermediate portion 140 and/or the right ventricular portion 130 can be cylindrical.

As shown in FIG. 1C, in some examples, when the expandable member 110 is in the second state, it can have a cylindrical shape, wherein each of the left ventricular portion cross-sectional area 122, the right ventricular portion cross-sectional area 132, and the intermediate portion cross-sectional area 142 are equal.

As shown in FIG. 1D, in some examples, when the expandable member 110 is in the second state, it can have an hourglass shape. When the expandable member has an hourglass shape, each of the left ventricular portion cross-sectional area 122 and the right ventricular portion cross-sectional area 132 can be greater than the intermediate portion cross-sectional area
142. Furthermore, the left ventricular portion 120 and the right ventricular portion 130 can taper towards the intermediate portion 140. In some examples, the expandable member 110 can be better anchored in the opening of the VSD 70 when both the left ventricular portion 120 and the right ventricular portion 130 are wider and/or larger than the intermediate portion 140.

As shown in FIG. 1E, in some examples, when the expandable member 110 is in the second state, it can have a dumbbell shape, wherein each of the left ventricular portion cross-sectional area 122 and the right ventricular portion cross-sectional area 132 can be greater than the intermediate portion cross-sectional area 142. One exemplary difference between the illustrated dumbbell shape and the hourglass shape shown in FIG. 1D is that the left ventricular portion 120 and the right ventricular portion 130 of the dumbbell shape do not taper towards the intermediate portion 140.

In some examples, when the expandable member 110 is in the first state, it is smaller than the size of the expandable member in the second state but can otherwise have the same overall shape of the expandable member 110 in the second state. For example, the expandable member 110 can have a mushroom shape in the first and second states (FIG. 1B), a cylindrical shape in the first and second states (FIG. 1C), an hourglass shape in the first and second states (FIG. 1D), or a dumbbell shape in the first and second states (FIG. 1E). When formed from a hydration-expandable material, the expandable member 110 can expand proportionally from the first state to the second state upon absorption of a fluid, such as blood, saline, contrast solution, etc. Moreover, when the expandable member 110 is in the first state, it can be deformed or radially compressed to fit inside a capsule or sheath of a delivery apparatus. Thus, in some examples, when the expandable member 110 is in the first state and loaded into a capsule of a delivery device, the expandable member 110 can be deformed and retained in a cylindrical shape. Although the expandable member 110 is illustrated in the various examples as having certain shapes, it should be understood that the expandable member 110 can have any other shape in the first state and the second state.

In some examples, the medical device 100 desirably is shaped such that it does not exert any clamping forces against tissue of the ventricular septum 40 in the left ventricle 20 and/or the right ventricle 30. In this manner, the medical device 100 can prevent or minimize contact with conductive tissue of the heart 10. For example, for the mushroom shaped device 100 shown in FIG. 1A, the right ventricular portion 130 is shaped to minimize or prevent contact with conductive tissue in the right ventricle 30. In some examples, the right ventricular portion 130 can be the same size as or slightly larger than the VSD 70. The mushroom shaped device 100 can remain anchored within the VSD 70 due to the fact that pressure in the left ventricle 20 is greater than the pressure in the right ventricle 30 during the cardiac cycle.

As noted above, the expandable member 110 can be larger in the second, larger state than in the first, smaller state. The ratio of the volume of the left ventricular portion in the second state to the volume of the left ventricular portion in the first state can range from two and one hundred, such as from two to ten, two to twenty-five, two to fifty, twenty-five to seventy-five, fifty to one hundred, seventy-five to one hundred, or ninety to one hundred. The ratio of the volume of the right ventricular portion in the second state to the volume of the right ventricular portion in the first state can range from two and one hundred, such as from two to ten, two to twenty-five, two to fifty, twenty-five to seventy-five, fifty to one hundred, seventy-five to one hundred, or ninety to one hundred. The ratio of the volume of the intermediate portion in the second state to the volume of the intermediate portion in the first state can range from two and one hundred, such as from two to ten, two to twenty-five, two to fifty, twenty-five to seventy-five, fifty to one hundred, seventy-five to one hundred, or ninety to one hundred. In some examples, the ratio of the volumes of left ventricular portion, the right ventricular portion, and/or the intermediate portion can be controlled. For example, any of these ratios can be controlled based on material selection, cross-linking density, the geometry of the expandable member 110, etc. In some examples, different portions of the expandable member 110 can be formed from different materials (for example, materials with different rates or coefficients of expansion) to more precisely control the expansion of the expandable member 110. In some examples, different portions of the expandable member 110 can have different sizes or geometries to more precisely control the expansion of the expandable member 110.

The expandable member 110 can be formed from any hydration-expandable material. In some examples, the hydration-expandable material can comprise a hydrogel. Example hydrogels can include, but are not limited to, polymeric hydrogels (such as alginate hydrogels and poly(NIPPAm-co-HEMA-co-MAPLA) hydrogels, etc.), polymer-cell complex hydrogels (such as α-CD/MPEG-PCL-MPEG hydrogels and ADSC-loaded ALG-TA-APTC hydrogels, etc.), polymer-cytokine complex hydrogels (such as PEG hydrogels combined with Shh and IL-10 growth factors, etc.), and/or polymer-protein complex hydrogels (such as protein-loaded microsphere hybrid hydrogels, alginate hydrogels, alginate-chitosan hydrogels, protein-loaded microsphere/hydrogel hybrid systems, etc.). In some examples, the hydration-expandable material can comprise polyethylene. In some examples, the hydration-expandable material can comprise a protein-based material. In some examples, the hydration-expandable material can comprise a polysaccharide-based material. In some examples, the hydration-expandable material can comprise a polyacrylate-based material. In some examples, the hydration-expandable material can comprise a polyvinyl alcohol-based material. In some examples, the hydration-expandable material can comprise an ethylene vinyl alcohol copolymer dissolved in dimethyl sulfoxide. In some examples, the hydration-expandable material can comprise a gelatin sponge material, for example, a gelatin sponge made from a porcine-derived gelatin or gelatin derived from other animals. In some examples, the gelatin sponge material is derived from animal skin, such as porcine skin. In some examples, the expandable member 110 is not formed from metal and/or does not comprise metal components that might potentially interact with a nearby anatomical structure. Thus, some examples of the expandable member 110 can be substantially or completely devoid of metal.

Any of the hydration-expandable material and/or material forming other components of the medical device 100 can be biocompatible. In some examples, any of these materials can optionally comprise a bioabsorbable material. Forming components from bioabsorbable material can beneficially allow tissue to reendothelialize around the medical device 100 (such as the expandable member 110), thereby allowing the medical device 100 to be absorbed or bio-absorbed by the body over time. In some examples, the biocompatible medical device 100 can promote tissue ingrowth.

In some examples, the expandable member 110 and/or other components of the medical device 100 can optionally comprise a radiopaque or echogenic material. Example radiopaque materials can include, but are not limited to, platinum, gold, palladium, and tantalum. In some examples, the radiopaque material can be in the form of a powder. The powder can be mixed with the hydration-expandable material before the hydration-expandable material is formed into the expandable member 110. In some examples, the radiopacity of the medical device 100 can be enhanced by hydrating the expandable member 110 and/or another portion of the medical device 100 with a solution of contrast and saline prior to use. In some examples, soaking the expandable member 110 and/or the medical device 100 in a pre-measured volume of 85% saline and 15% contrast solution can provide observable radiopacity to the medical device 100.

In some examples, the medical device 100 can optionally comprise a covering 150 disposed on at least a portion of an outer surface of the expandable member 110. In some examples, the covering 150 can be disposed over the entirety of the outer surface of the expandable member 110. The covering 150 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc.

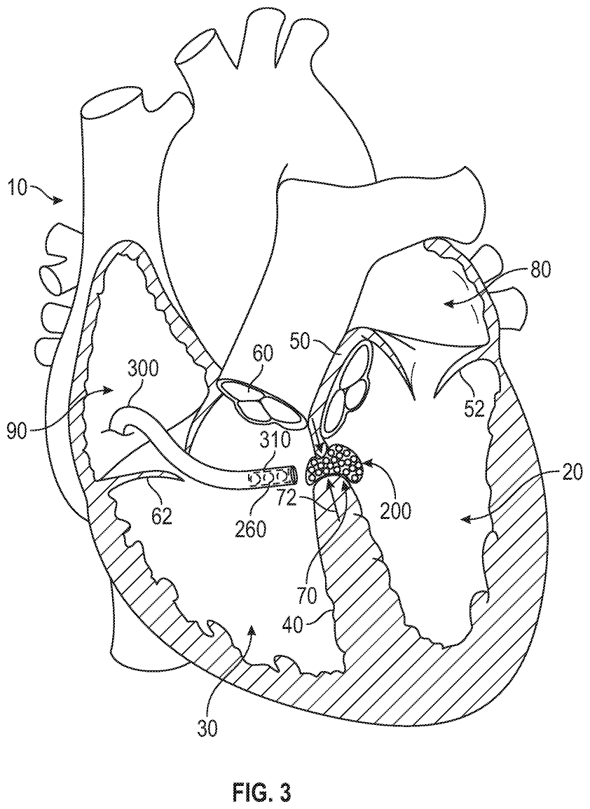

In some examples, where the covering 150 comprises a fabric, the covering 150 can have a single jersey knit construction or a knitted construction with a knit and tuck stitch combination. Now referring to FIG. 4, an exemplary fabric 450 for forming the covering 150 has a single jersey knit construction comprising a first yarn 410 and a second yarn 420. The first yarn 410 can comprise a flat, twisted, or textured yarn. The second yarn 420 can comprise an elastic yarn. In some examples, the first yarn 410 can have a denier range from 10 denier to 200 denier. In some examples, the first yarn 410 can have a filament count from 10 to 96. The second yarn 420 can be formed from an implantable elastomeric polymer, such as thermoplastic polyurethane (TPU), a polyolefin-based elastomer, etc. In some examples, the second yarn 420 can have an elongation at break property from 50% to 700%. In some examples, the second yarn 420 can have a tensile strength of at least 2.5 grams per denier.

In some examples, the fabric 450 can alternatively comprise a non-elastic second yarn 420. In such examples, the textured first yarn 410 can have hot-air shrinkage in a range from 10% to 70% to create additional stretchability in the covering 150. In some examples, the first yarn 410 can be formed from a polyester (such as polyethylene terephthalate (PET)) and/or a thermoplastic polymer (such as nylon). In some examples, the first yarn 410 can be heat treated by heating the first yarn 410 to a temperature in a range from 100° C. to 200° C. for a duration in a range from 5 minutes to 30 minutes to confer the desired hot-air shrinkage properties on the first yarn 410.

In some examples, the illustrated fabric 450 can be knitted with a density from 20 wales per inch to 60 wales per inch and from 10 courses per inch to 60 courses per inch.

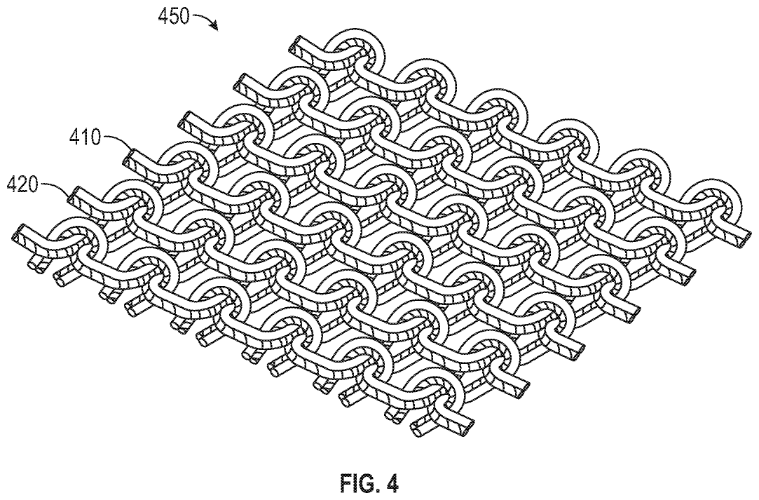

Now referring to FIG. 5, another exemplary fabric 550 for forming the covering 150 has a knitted construction comprising a main yarn 510 and a plated yarn 520. The main yarn 510 and the plated yarn 520 are knitted to form a combination of tuck stitches 530 and knit stitches 540. In some examples, the main yarn 510 can be formed from an elastomeric polymer (such as TPU). In some examples, the main yarn 510 can be a fine yarn. In some examples, the plated yarn 520 can be formed from an elastomeric polymer (such as TPU). In some examples, the plated yarn 520 can have a linear mass fiber density in a range from 10 denier to 200 denier. As shown, tuck stitches 530 are formed on every other course, and subsequent tuck stitches 530 along a course are formed on every other wale. To construct a course comprising both tuck stitches 530 and knit stitches 540, the plated yarn 520 can be fed at a high level and can be received and knitted by every other needle, and the main yarn 510 can be fed at a low level and can be received and knitted by every needle.

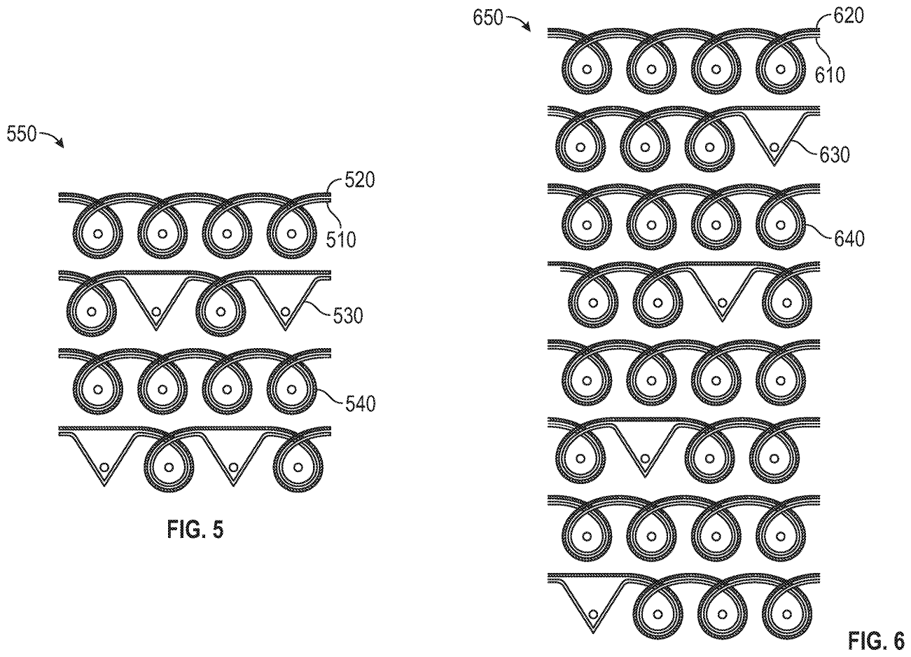

Now referring to FIG. 6, another exemplary fabric 650 for forming the covering 150 has a knitted construction comprising a main yarn 610 and a plated yarn 620 forming a combination of tuck stitches 630 and knit stitches 640. As shown, tuck stitches 630 are formed on every other course, and tuck stitches 630 are advanced one wale at a time in subsequent courses.

Now referring back to FIGS. 1A-1E, in some examples, the covering 150 can comprise a fabric without interlaced yarns or fibers or randomly interlaced yarns or fibers (sometimes referred to as non-woven fabrics), such as felt or an electrospun fabric. Exemplary materials that can be used for forming such fabrics (with or without interlaced yarns or fibers) include, without limitation, nylons, polyesters, polyester-PET, polyethylene (PET), ultra-high molecular weight polyethylene (UHMWPE), polypropylenes, polytetrafluoroethylene (PTFE), expanded polytetrafluoroethylene (ePTFE), polyamides, etc.

In some examples, the covering 150 can comprise a non-textile or non-fabric material, such as a film made from any of a variety of polymeric materials, such as PTFE, PET, polypropylene, polyester, polyamide, PTFE, expanded PTFE, nylon, polyetheretherketone (PEEK), polyurethane (such as thermoplastic polyurethane (TPU)). In some examples, the covering 150 can be configured to promote tissue ingrowth and/or prevent rejection of the medical device 100 (for example, by forming the covering 150 from a biocompatible material). In some examples, the covering 150 can be bioabsorbable.

The medical device 100 can be substantially or completely devoid of metal. For example, as shown in FIGS. 1A-1E, the medical device 100 can lack the metal frame of typical medical devices. In some examples, the medical device 100 can lack other components formed of partially, substantially, or entirely of metal that are typically included in known or conventional implantable medical devices (such as fasteners, struts, electronics, housings, frames, etc.). Furthermore, as previously discussed, the expandable member 110 can be substantially or completely devoid of metal. Thus, some examples of the medical device 100 as a whole can be substantially or completely devoid of metal. In some examples, eliminating the metal frame and/or other metal components can beneficially minimize the likelihood that the medical device 100 will interact with the nearby heart valve (for example, the aortic valve 50 and/or the pulmonary valve 60 adjacent the perimembranous VSD 70). In some examples, eliminating the metal frame and/or other metal components can beneficially reduce or eliminate the need to clock the medical device 100 during implantation. In some examples, eliminating the metal frame and/or other metal components can beneficially further reduce the risk of septal tissue erosion and/or heart block.

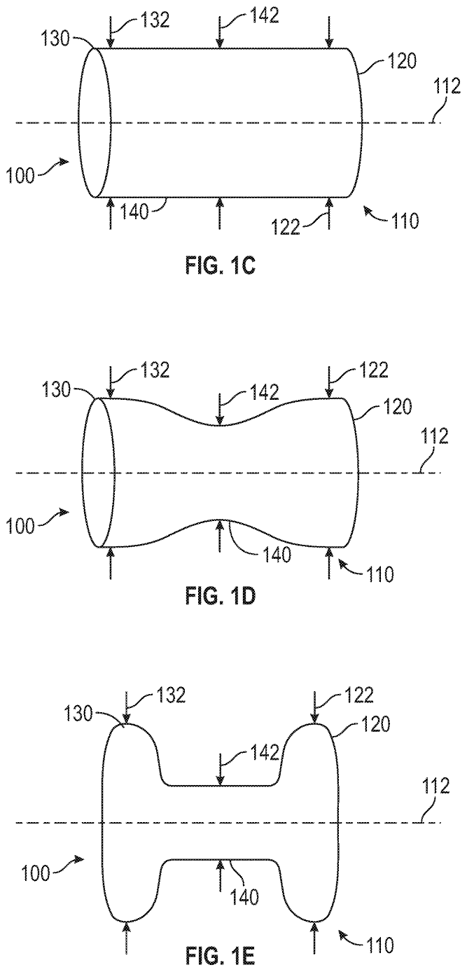

FIG. 2A illustrates an implantable medical device 200 implanted in the subject's heart 10, according to one example. One exemplary difference between the implantable medical device 200 (which is also referred to as a “medical device” or an “occlusion device”) and the implantable medical device 100 of FIGS. 1A-1E is that the implantable medical device 200 can comprise an expandable container 210 defining an inner cavity therein. The expandable container 210 can be configured to expand from a first state (which is also referred to herein as a “compressed state,” and/or a “delivery state”) to a second state (which can also be referred to herein as an “expanded state,” and/or a “deployed state”) when a hydration-expandable material disposed within the inner cavity of the expandable container 210 is hydrated upon contact with a fluid, such as blood, water, saline, contrast solution, etc. As shown, the hydration-expandable material is formed into a plurality of expandable beads 260 (which are also referred herein as “hydration-expandable beads,” “pellets,” and/or “pearls”). However, it should be understood that the hydration-expandable material does not need to be formed into beads and can have any other form (for example, a powder, a liquid, a paste, or any combination thereof). In some examples, in lieu of or in addition to the beads 260 the expandable container 210 can be configured to expand from the first state to the second state when an inflation fluid (such as a gas or a liquid) is introduced into the cavity of the expandable container 210.

In some examples, the expandable container 210 can comprise a sack, a balloon, or a basket, and can have a braided, woven, or knitted construction configured to retain the plurality of expandable beads 260 in the cavity therein. In some examples, the container 210 can be formed from a fabric having the construction shown in FIGS. 4, 5, or 6. The expandable container 210 can be at least partially porous, such that blood, water, saline, contrast solution, or other fluids can enter the cavity of the expandable container 210 and hydrate the plurality of expandable beads 260 disposed therein. In some examples, the rate of expansion of the plurality of expandable beads 260 can be correlated, related, or proportional with the porosity of the expandable container 210. In some examples, the porosity of the expandable container 210 can be selected to control the rate of expansion of the expandable beads 260. For example, pluralities of expandable beads 260 can expand at a faster rate in expandable containers 210 with higher porosities (which allows fluid to enter the cavity of the expandable container 210 at a faster rate) as compared with expandable containers 210 with lower porosities. In some examples, the porosity of the expandable container 210 can be selected to allow for quick expansion of the plurality of expandable beads 260 without compromising the structural integrity of the expandable container 210. The expandable container 210 can be flexible or deformable to accommodate the expansion of the plurality of expandable beads 260 from a first, compressed state to a second, expanded state. In some examples, the expandable container 210 can further comprise an opening through which the plurality of expandable beads 260 can be inserted. The opening can be closable or sealable to retain the plurality of expandable beads 260 within the cavity after implantation.

The expandable container 210 can define a left ventricular portion 220, a right ventricular portion 230, and an intermediate portion 240 aligned along a central longitudinal axis 212. The left ventricular portion 220 can be configured to be disposed in the left ventricle 20 when the expandable container 210 is in the second state and implanted in the heart 10. The right ventricular portion 230 can be configured to be disposed in the right ventricle 30 when the expandable container 210 is in the second state and implanted in the heart 10. The intermediate portion 240 can be disposed between the left ventricular portion 220 and the right ventricular portion 230 of the expandable container 210.

As shown in FIG. 2B, the left ventricular portion 120 can define a left ventricular portion cross-sectional area 222, the right ventricular portion 230 can define a right ventricular portion cross-sectional area 232, and the intermediate portion 140 can define an intermediate portion cross-sectional area 242. Each cross-sectional area 222, 232, and 242 can extend in a radial direction substantially perpendicular to the central longitudinal axis 212 of the medical device 200 and can define the largest cross-sectional area of the respective portion of the medical device 200.

In some examples, the expandable container 210 can comprise a plurality of compartments. In some examples, the expandable container 210 can comprise a first compartment corresponding to the left ventricular portion 220 of the expandable container 210 and a second compartment corresponding to the right ventricular portion 230 of the expandable container 210. In some examples, the expandable container 210 can comprise a third compartment corresponding to the intermediate portion 240 of the expandable container 210. The compartments can be arranged or aligned along the central longitudinal axis 212. In some examples, a first set of compartments can be filled with a first number of the plurality of expandable beads 260. In some examples, a second set of compartments can be filled with a second number of the plurality of expandable beads 260. For example, the first compartment can be filled prior to or during implantation with a greater number of expandable beads 260 than the second compartment, such that the left ventricular portion cross-sectional area 222 becomes larger than the right ventricular portion cross-sectional area 232 when the plurality of expandable beads 260 are hydrated and expandable container 210 expands to the second state.

Some examples of the expandable container 210 can be formed from a polymer, such as any combination of nylon, polyesters, polyethylenes (for example, high density polyethylene and/or low-density polyethylene), polypropylenes, polypropylene copolymer, polytetrafluoroethylene, and expanded polytetrafluoroethylene. The container 210 can be formed from any of the materials mentioned above for the covering 150.

Each of the plurality of expandable beads 260 can comprise a pellet, bead, pearl, or other structure formed from a hydration-expandable material (for example, any of the hydration-expandable materials described in the present disclosure). Thus, each of the plurality of expandable beads 260 can be in a first state (for example, a dehydrated, compressed, or delivery state) prior to implantation in the subject's heart 10 and, as shown, can be expanded to a second state (for example, a hydrated, expanded, or deployed state) during implantation such that the expandable member 110 occludes the opening of the VSD 70.

Each of the plurality of expandable beads 260 can have any shape when hydrated or expanded. As shown, each of the plurality of expandable beads 260 can have a substantially spherical shape when in the hydrated or expanded state. In some examples, each of the plurality of expandable beads 260 in the second state can have any shape, such as the shape of a cube, a cone, a pyramid, a rod, or combinations thereof. In some examples, the medical device 200 can comprise a first set of the plurality of expandable beads 260 having a first shape and a second set
of the plurality of expandable beads 260 having a second shape when the plurality of expandable beads 260 are in the second, hydrated state. Each of the plurality of expandable beads 260 can have any shape in the first, dehydrated state.

As shown, each of the plurality of expandable beads 260 can have the same size (for example, the same diameter, cross-sectional area, and/or volume) in the second state. However, some examples of the medical device 200 can comprise a first set of the plurality of expandable beads 260 having a first size in the second, hydrated state and a second set of the plurality of expandable beads 260 having a second, different size in the second, hydrated state. In some examples, smaller ones of the plurality of expandable beads 260 can fill gaps between larger ones of the plurality of expandable beads 260, thereby increasing the packing efficiency of the plurality of expandable beads 260 and beneficially resulting in better, more complete occlusion of the VSD 70.

In some examples, each of the plurality of expandable beads 260 can be formed from any of the hydration-expandable materials described in the present disclosure. In some examples, any number of the plurality of expandable beads 260 can optionally comprise any of the biocompatible materials, bioabsorbable materials, and/or radiopaque materials described in the present disclosure.

Now referring back to FIG. 2A, the plurality of expandable beads 260 can be hydrated within the cavity of the expandable container 210 such that the left ventricular portion cross-sectional area 222 of the expandable container 210 in the second state can be greater than the VSD planar area 72 to occlude the opening of the VSD 70 on the left ventricular side. In some examples, the right ventricular portion cross-sectional area 232 of the expandable container 210 in the second state can additionally be wider than the VSD planar area 72 to better anchor the medical device 200 at the VSD 70. As shown, the intermediate portion cross-sectional area 242 of the expandable container 210 can be less than each of the left ventricular portion cross-sectional area 222 and the right ventricular portion cross-sectional area 232 when the expandable container 210 is in the second state. In some examples, the expandable container 210 can have any shape, such as any of the cylinder, dumbbell, hourglass, bell, mushroom, or spherical shapes described in the present disclosure.

In some examples, each of the plurality of expandable beads 260 can be coupled to a string or cord to facilitate filling the container 210 with the beads.

### Delivery Techniques

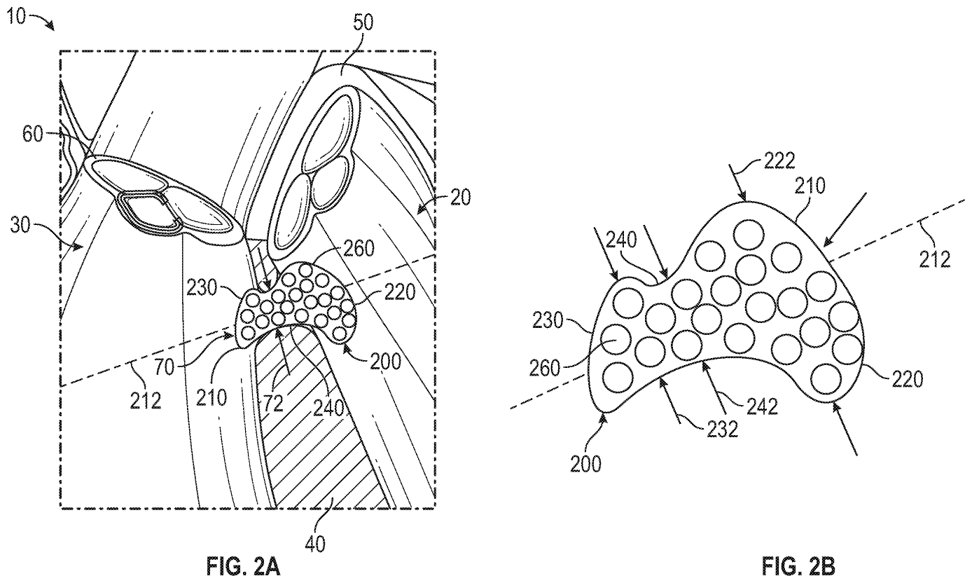

FIG. 3 illustrates a method for delivering the medical device 200 into the subject's heart 10, according to one example. The method can comprise a transcatheter procedure using a delivery apparatus 300 (which is also referred to herein as a “catheter” and/or a “delivery catheter”). Although the illustrated method is used to deliver the medical device 200, the method can be used to deliver the medical device 100 or any other one of the disclosed medical devices into the subject's heart 10. The disclosed delivery methods are not intended to be limited. Any of the medical devices disclosed herein can be implanted using any of various delivery procedures and delivery devices known in the art.

With reference to FIG. 3, the medical device 200 can be delivered to the VSD 70 by coupling the medical device 200 to a distal end portion of the delivery apparatus 300. Alternatively, the medical device 200 can be positioned inside of the distal end portion of the delivery apparatus 300. The delivery apparatus can be inserted into a femoral vein or jugular vein, advanced into and through one of the inferior vena cava or superior vena cava, and into the right atrium 90. The medical device 200 coupled to the distal end of the delivery apparatus 300 can further be advanced through the native tricuspid valve 62, into the right ventricle 30, and towards the implantation site at the opening of the VSD 70.

In some examples, the medical device 200 can be delivered by advancing the distal end portion of the delivery apparatus 300 into the left atrium 80 of the heart 10, through the mitral valve 52, into the left ventricle 20, and towards to the implantation site at or near the opening of the VSD 70.

In some examples, the medical device 200 can be delivered by inserting the delivery apparatus 300 into a femoral artery and advancing the distal end portion of the delivery apparatus 300 into and through the descending aorta, around the aortic arch, and through the ascending aorta. The medical device 200 can further be advanced across the aortic valve 50, into the left ventricle 20 of the heart 10, and towards the implantation site at the opening of the VSD 70.

In any of the disclosed delivery approaches, the delivery apparatus 300 can be advanced over a guidewire previously inserted into the subject's vasculature. In any of the disclosed delivery approaches, the delivery apparatus 300 can comprise a steering mechanism to steer or adjust the distal end portion of the delivery apparatus 300.

After being advanced into the heart by the delivery apparatus, the medical device 200 can be positioned within the VSD 70. In some examples, the cavity of the expandable container 210 can be at least partially filled with the plurality of expandable beads 260 after the expandable container 210 is situated at the implantation site. For example, during delivery, the container 210 can be empty (devoid of any beads 260) and then filled with a desired number of beads 260 after the container is adjacent or within the VSD 70. In some examples, the body of the expandable container 210 can be at least partially filled or completed filled with the plurality of expandable beads 260 prior to implantation.

In some examples, the plurality of expandable beads 260 can be introduced into the cavity of the expandable container 210 the via the delivery apparatus 300. As shown, the delivery apparatus 300 can comprise a chamber or lumen 310 disposed at least along the distal end portion thereof. The chamber 310 can be configured to hold the plurality of expandable beads 260 in the first, compressed state. Once the expandable container 210 is situated at the implantation site, the plurality of expandable beads 260 can be deployed from the chamber 310, for example, using a plunger or pusher shaft, to at least partially fill the cavity of the expandable container 210 with the plurality of expandable beads 260. In some examples, the chamber 310 of the delivery apparatus 300 can be substantially devoid of blood, water, saline, contrast solution, and/or other fluids prior to the advancement of the beads 260 to prevent the plurality of expandable beads 260 from prematurely expanding from the first state to the second state. The plurality of expandable beads 260 hydrate and expand as they exit the chamber 310 of the delivery apparatus 300. In some examples, the beads can be coupled to a string or cord (similar to a string of pearls) to facilitate advancement of the beads from the delivery apparatus into the container 210, and to allow for the beads to be retracted back into the delivery apparatus 300 as desired.

In some examples, a user of the delivery apparatus 300 can control the advancement of the plurality of expandable beads 260 from the delivery apparatus 300. For example, the user can actuate a control interface on a handle of the delivery apparatus 300 to control the rate at which the plurality of expandable beads 260 exit and/or re-enter the chamber 310.

In some examples, the user can determine the number of plurality of expandable beads 260 to be advanced from the chamber 310 such that the medical device 200 occludes the opening of the VSD 70. In some examples where the expandable beads 260 or other components of the medical device 200 are radiopaque, the user can use an X-ray system, a fluoroscopy system, or any other imaging system to view the medical device 200 positioned in the subject's heart 10. The user can determine, based on the imaging system, whether the expandable member 110 has been sufficiently expanded such that it occludes the opening of the VSD 70. For example, the user can determine, based on measurements derived from a fluoroscopic image, whether the expandable member 110 has been sufficiently expanded such that it occludes the opening of the VSD 70. In some examples, the imaging system can automatically determine whether the expandable member 110 has been sufficiently expanded such that it occludes the opening of the VSD 70. In some examples, the imaging system can be operably coupled to the delivery apparatus 300 to automatically expand the expandable member 110 based on this determination.

In some examples, the medical device 200 can be recovered and removed from the subject's body after the medical device 200 has been implanted in the subject's heart 10 (for example, in a subsequent medical procedure or at a later stage in the same medical procedure). For example, the distal end of the delivery apparatus 300 can be aligned with and coupled to the implanted medical device 200. The medical device 200 and the delivery apparatus 300 can subsequently be retracted in unison in the proximal direction to recover the medical device 200. This optional stage of the method can beneficially allow for the medical device 200 to be repositioned or moved after implantation.

Although the examples of medical devices disclosed herein have been described in connection with repairing a ventricular septal defect, any of the medical devices disclosed herein can be implanted within a septal defect in the atrial septum.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Simulation

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. An implantable device for occluding an opening in a septum of a heart can include an expandable member that is configured to expand from a first state to a second state upon contact with fluid.
Example 2. The implantable device of any example herein, particularly example 1, wherein the expandable member can be substantially devoid of metal.
Example 3. The implantable device of any example herein, particularly example 1, wherein the expandable member can be completely devoid of metal.
Example 4. The implantable device of any example herein, particularly any one of examples 1-3, wherein the expandable member can include a hydrogel.
Example 5. The implantable device of any example herein, particularly any one of examples 1-4, wherein the expandable member can include a gelatin sponge.
Example 6. The implantable device of any example herein, particularly any one of examples 1-5, wherein the expandable member can be bioabsorbable.
Example 7. The implantable device of any example herein, particularly any one of examples 1-6, wherein the expandable member can be tissue ingrowth promoting.
Example 8. The implantable device of any example herein, particularly any one of examples 1-7, wherein the expandable member can be radiopaque.
Example 9. An implantable device for occluding an opening in a septum of a heart can include a non-metal expandable body expandable between a first state and a second state.
Example 10.The implantable device of any example herein, particularly example 9, wherein the expandable body can have a first cross-sectional area in the first state and can have a second cross-sectional area in the second state.
Example 11.The implantable device of any example herein, particularly any one of examples 9-10, wherein the second cross-sectional area of the expandable body can be greater than a planar area of the opening in the septum.
Example 12.The implantable device of any example herein, particularly example 11, wherein the first cross-sectional area of the expandable body can be less than the planar area of the opening in the septum.
Example 13.The implantable device of any example herein, particularly any one of examples 9-12, wherein the expandable body can have a mushroom shape in the second state.
Example 14.The implantable device of any example herein, particularly any one of examples 9-12, wherein the expandable body can have a dumbbell shape in the second state.
Example 15.The implantable device of any example herein, particularly any one of examples 9-12, wherein the expandable body can have an hourglass shape in the second state.
Example 16.The implantable device of any example herein, particularly any one of examples 9-15, can further include a covering disposed on at least a portion of an outer surface of the expandable body.
Example 17.The implantable device of any example herein, particularly example 16, wherein the covering can be configured to promote tissue ingrowth.
Example 18.An implantable medical device for occluding an opening of a ventricular septal defect of a heart can include an expandable container defining a cavity therein, and a plurality of expandable beads disposed within the cavity of the expandable container and configured to be hydrated to an expanded state.
Example 19.The implantable medical device of any example herein, particularly example 18, wherein the expandable container can include a braided, woven, or knitted layer.
Example 20.The implantable medical device of any example herein, particularly any one of examples 18-19, wherein the expandable container can be formed from a biocompatible textile.
Example 21.The implantable medical device of any example herein, particularly any one of examples 18-20, wherein the expandable container can include first and second compartments.
Example 22.The implantable medical device of any example herein, particularly example 21, wherein the first compartment can be configured to be filled with a first number of the plurality of expandable beads and the second compartment can be configured to be filled with a second, lesser number of the plurality of expandable beads.
Example 23.The implantable medical device of any example herein, particularly any one of examples 21-22, wherein the first compartment can be configured to be disposed in a left ventricle of the heart and the second compartment can be configured to be disposed in a right ventricle of the heart when the implantable medical device is implanted in the heart.
Example 24.The implantable medical device of any example herein, particularly any one of examples 18-23, wherein the plurality of expandable beads can include a first set of expandable beads having a first size in the expanded state and a second set of expandable beads having a second size in the expanded state.
Example 25.The implantable medical device of any example herein, particularly any one of examples 18-24, wherein the expandable container can have a porosity, and wherein the rate of expansion of the plurality of expandable beads can be correlated with the porosity of the expandable container.
Example 26. A method of implanting a medical device in a heart can include advancing a catheter coupled to the medical device towards a ventricular septal defect of the heart; using the catheter, positioning the medical device at the ventricular septal defect; and advancing a plurality of expandable beads from the catheter into a cavity of the medical device.
Example 27.The method of any example herein, particularly example 26, can further include, prior to advancing the plurality of expandable beads, determining a number of the plurality of expandable beads to advance from the catheter.
Example 28.The method of any example herein, particularly example 27, wherein the determination can be based on measurements derived from a fluoroscopic image.
Example 29.The method of any example herein, particularly any one of examples 26-28, can further include, after advancing a plurality of expandable beads from the catheter into a cavity of the medical device: coupling the catheter to the medical device; and retracting the catheter and the medical device in unison in a proximal direction.
Example 30.The implantable device of any example herein, particularly any one of examples 1-29, wherein the implantable device can be sterilized.

The features described herein with regard to any example can be combined with other features described in any one or more of the other examples, unless otherwise stated. For example, any one or more features of one implantable medical device can be combined with any one or more features of another implantable medical device. In some examples, any one or more features of one method can be combined with any one or more features of another method.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. An implantable device for occluding an opening in a septum of a heart comprising an expandable member configured to expand from a first state to an expanded second state upon contact with fluid, the expandable member having a first end portion, an intermediate portion, and a second end portion arranged along a longitudinal axis extending through the expandable member, wherein the intermediate portion is disposed between the first end portion and the second end portion, wherein the expandable member is asymmetrical with respect to a transverse axis that extends through the intermediate portion and is perpendicular to the longitudinal axis with the first end portion having a smaller cross-sectional area in a plane perpendicular to the longitudinal axis than the second end portion when the expandable member is in the expanded second state.

2. The implantable device of claim 1, wherein the first end portion is configured to be positioned in the right ventricle when the implantable device is implanted in the opening in the septum, and wherein the first end portion is shaped to minimize contact with conductive tissue in the right ventricle.

3. The implantable device of claim 2, wherein the cross-sectional area of the first end portion in the expanded second state is equal to or smaller than a planar area of the opening, and wherein the first end portion in the expanded second state is held in place within the opening by pressure in the left ventricle acting upon the second end portion.

4. The implantable device of claim 2, wherein the cross-sectional area of the first end portion in the expanded second state is greater than a planar area of the opening to assist in anchoring the expanded member in the opening.

5. The implantable device of claim 1, wherein the second end portion in the expanded second state has a cross-sectional area in a plane perpendicular to the longitudinal axis that is greater than a planar area of the opening.

6. The implantable device of claim 1, further comprising a covering disposed on at least a portion of an outer surface of the expandable member.

7. The implantable device of claim 6, wherein the covering is configured to promote tissue ingrowth.

8. The implantable device of claim 6, wherein the cover comprises a knitted construction or a non-woven fabric.

9. The implantable device of claim 1, wherein the expandable member is mushroom shaped with the second end portion having a larger cross-sectional area than the first end portion.

10. The implantable device of claim 1, wherein the expandable member is substantially devoid of metal.

11. The implantable device of claim 1, wherein the expandable member comprises a hydrogel.

12. The implantable device of claim 1, wherein the expandable member is bioabsorbable.

13. The implantable device of claim 1, wherein the expandable member is radiopaque.

14. An implantable medical device for occluding an opening of a ventricular septal defect of a heart comprising:
an expandable container defining a cavity therein, wherein the expandable container comprises at least first and second compartments; and
a plurality of expandable beads disposed within the first and second compartments of the cavity of the expandable container and configured to be hydrated to an expanded state,
wherein the first compartment is selectively filled with a first number of the plurality of expandable beads and the second compartment is selectively filled with a second, lesser number of the plurality of expandable beads.

15. The implantable medical device of claim 14, further comprising an intermediate compartment positioned between the first and second pockets.

16. The implantable medical device of claim 14, wherein the expandable container comprises a braided, woven, or knitted layer that is biocompatible.

17. A method of implanting a medical device in a septum of a heart, the medical device comprising first and second end portions and an intermediate portion between the first and second end portions, wherein the first end portion, the intermediate portion, and the second end portion are arranged along a longitudinal axis of the medical device, the method comprising:
advancing a catheter coupled to the expandable member towards a ventricular septal defect of the heart while the medical device is in a first, unexpanded state;
using the catheter, positioning the medical device in the ventricular septal defect with the first end portion extending into the right ventricle and the second end portion extending into the left ventricle;
advancing a plurality of expandable beads from the catheter into a cavity of the medical device; and
allowing the expandable member to contact fluid and expand from the first state to an expanded second state such that the second end portion in the left ventricle has a cross-sectional area in a plane perpendicular to the longitudinal axis that is larger than the ventricular septal defect, wherein the medical device, in the expanded second state, is asymmetrical with respect to the ventricular septal defect.

18. The method of claim 17, further comprising, prior to advancing the plurality of expandable beads, determining a number of the plurality of expandable beads to advance from the catheter.

19. The method of claim 18, wherein the determination is based on measurements derived from a fluoroscopic image.

20. The method of claim 17, wherein the cavity comprises at least first and second compartments, and wherein advancing the plurality of expandible beads from the catheter into the cavity comprises positioning a first number of the plurality of expandible beads in the first compartment and positioning a second, lesser number of the plurality of expandible beads in the second compartment, and wherein the first compartment becomes larger than the second compartment when the expandable member is expanded from the first state to the second state.

21. The method of claim 17, wherein when the medical device is in the expanded second state, the medical device does not exert a clamping force against tissue of the ventricular septum.
